(19) United States
(12) Patent Application Publication
(10) Pub. No.: US 2007/0179503 A1
(43) Pub. Date: Aug. 2, 2007

(54) Cross-coupled vertebral stabilizers incorporating spinal motion restriction

(76) Inventor: Bret A. Ferree, Cincinnati, OH (US)

Correspondence Address: O'MELVENY & MYERS LLP, 610 NEWPORT CENTER DRIVE, 17TH FLOOR, NEWPORT BEACH, CA, 92660, US

(21) Appl. No.: 11/703,933

(22) Filed: Feb. 7, 2007

Related U.S. Application Data
(63) Continuation of application No. 10/152,485, filed on May 21, 2002.
Application No. 10/152,485 is a continuation-in-part of application No. 09/841,324, filed on Apr. 24, 2001, now Pat. No. 6,423,065.
Application No. 09/841,324 is a continuation-in-part of application No. 09/513,127, filed on Feb. 25, 2000, now Pat. No. 6,248,106.

(51) Int. Cl. A61F 2/30

(52) U.S. Cl. 606/61

(57) ABSTRACT

A method for stabilizing upper and lower spinal vertebrae having a disc space situated therebetween is described. First and second fasteners are inserted into the upper vertebra. Third and fourth fasteners are inserted into the lower vertebra, such that the first and third fasteners are substantially vertically aligned and the second and fourth fasteners are substantially vertically aligned. The first and third fasteners are connected with a first elongate element. The second and fourth fasteners are connected with a second elongate element. The first and fourth fasteners are connected with a third elongate element. The second and third fasteners are connected with a fourth elongate element.

### REFERENCE TO RELATED APPLICATION

This application is a continuation of U.S. patent application Ser. No. 10/152,485, filed May 21, 2002, which is a continuation-in-part of U.S. patent application Ser. No. 09/841,324, filed Apr. 24, 2001, now U.S. Pat. No. 6,423,065, which is a continuation-in-part of U.S. patent application Ser. No. 09/513,127, filed Feb. 25, 2000, now U.S. Pat. No. 6,248,106, the entire content of each application being expressly incorporated herein by reference in its entirety.

### FIELD OF THE INVENTION

This invention relates generally to orthopedic spinal surgery and, in particular, to vertebral fixation methods and apparatus which provide multi-dimensional stability and apply compressive forces to enhance fusion.

### BACKGROUND OF THE INVENTION

In surgeries involving spinal fixation, interbody cages are often used to restore disc space height, serve as a conduit for bone graft, and to help immobilize vertebrae undergoing fusion. Distracting the disc space prior to cage insertion restore disc space height. Distraction serves two important functions. First, it can decrease pressure on spinal nerves by increasing the size of the intervertebral foramen. Second, distraction increases tension on the annulus fibrosis which, in turn, increases the stability of the vertebra-cage-vertebra construct.

Presumably the annular tension decreases with time, thus weakening the construct. Furthermore, the annulus is weakened in many patients with severe degenerative disc disease. Given these and other deficiencies with annular tension, additional fixation is frequently added to increase the rigidity of the vertebra-cage combination.

Currently such additional fixation is inserted onto or into the posterior aspect of the spine. Thus, patients who have cages inserted from an anterior approach must undergo a second operation from the posterior aspect of the body. As might be expected, the second surgery increases patient morbidity, insurance costs, and delays return to work.

There are two ways to insert supplemental fixation through the same incision. One technique uses the interbody cages disclosed in my co-pending U.S. patent application Ser. No. 09/454,908, the entire contents of which are incorporated herein by reference. Posterior insertion allows the addition of supplemental fixation through the same incision.

A second solution employs fixation inserted through the anterior aspect of the spine. With known anterior lumbar spine fixation techniques, a combination of screws and rods or plates are inserted on the lateral side of the vertebrae from an anterior or lateral approach. The fixation is placed on the lateral aspect of the spine to avoid the aorta. Previous metal devices placed under the aorta have lead to aneurysms in some cases (Dunn Device). Unfortunately, a few patients have died from rupture of the aneurysms.

Lateral fixation is not ideal with interbody cages. First, lateral fixation cannot be used at the L5-SI level. The iliac arteries cross the L5-S1 level anteriorly and laterally. Second, the vascular anatomy of many patients does not permit lateral fixation at the L4-L5 level. The majority of cages are inserted at the L4-L5 and L5-S1 levels. Third, cages are generally inserted in a directly anterior-to-posterior fashion with the patient in a supine position. Lateral instrumentation is difficult if not impossible in most patients in the supine position.

The system described in U.S. Pat. No. 5,904,682 uses two flat plates applied to screws placed bilaterally on either side of the disc space. The system does not use cables or diagonal bracing to resist rotational forces. In U.S. Pat. No. 4,854,304 screws laced in the side of the vertebral bodies are connected from a lateral approach. The screws are connected with a threaded rod. In 1964, A. F. Dwyer described a system using a single cable to connect screws placed on the lateral portion of the vertebral bodies. Dr. Dwyer connected a series of screws with one screw per vertebral body. The arrangement described in U. S. Pat. No. 4,854.304 is similar to Dr. Dwyer's system, but the cable is replaced with a threaded rod. Dr. Ziekle modified Dr. Dwyer's system in 1975, as set forth in Pat. No. 4,854,304.

Cables and tensioning devices are also well known in orthopedic spine surgery. References that use cables include U.S. Pat. Nos. 4,966,600; 5,423,820; 5,611,801; 5,702,399; 5,964,769; 5,997,542. None use diagonal members to enhance compression and resist lateral movement, however.

My U.S. Pat. No. 6,248,106 is directed to spinal stabilization mechanisms operative to prevent lateral bending, extension, and rotation at the disc space. Broadly, the mechanism includes two or more anchors at each vertebral level, and links for each anchor at each level to both anchors at the other level, resulting in a cross-braced arrangement.

In the preferred embodiment, the mechanism uses screws for placement in the vertebral bodies and cables are used to connect the screws. The cables pull the screws together, applying compression across the disc space. Bone graft, cages, or distracting plugs and the device to enhance fusion area would fill or cross the disc space. The bone graft, cages, etc. within the disc space are preferably used to resist compression.

The device may be used in the cervical, thoracic, or lumbar spine. The device is preferably placed anteriorly, but could also be used posteriorly, with the screws directed through the vertebral body pedicles. The various components may be constructed of titanium, stainless steel, polymers, or a combination of such materials.

The anchors preferably include a post protruding from the vertebra, and a cable-holders which fits over the post. The post may be threaded, in which case a nut would be used to tighten the holders, or the cable holders may be allowed to rotate, depending upon the position and/or application of the fasteners. The cable holders may use tunnels, tubes or outer grooves to the hold the cables in position. Devices may also be added to keep the links from crossing one another where they cross.

My U.S. patent application Ser. No. 09/841,324 discloses a refinement comprising a cam-operated cable-holding connector which may be used for vertebral alignment and other applications. The connector includes a lower screw portion configured to penetrate into a vertebrae, thereby leaving an exposed portion. A cable-holding mechanism attached to the exposed portion is operable between a first state, wherein one or more cables may be readily dressed therepast, and a second state, wherein a portion of the mechanism is rotated or otherwise physically manipulated to lock the one or more of the cables into position.

In the case of vertebral alignment, the lower screw portion is preferably a pedicle screw, and the mechanism includes a first body having an interrupted side wall with an inner surface, and a second body having a rotatable cam. In this case, the mechanism facilitates a first state, wherein the relationship between the cam and the inner surface of the side wall is such that the cables pass therethrough, and a second state, wherein the cam is turned so as to retain the one or more cables against the inner wall of the side wall.

Pedicle screws are generally connected by solid rods or plates in an attempt to eliminate spinal motion. Eliminating spinal motion helps the vertebrae fuse together. A few inventors have connected pedicle screws with rubber, elastic, or fibrous materials to dampen or restrict spinal motion. These inventors have postulated low back pain is caused by abnormal movements and/or pressure across the facet joints.

Initially, the pedicle screws were connected by fibrous bands to limit flexion of the spine (distraction of the posterior portion of the vertebrae). The devices were improved by covering the fibrous bands with rubber sleeves which help dampen the forces on the facets that occurs with spinal extension. That is, the rubber sleeves help prevent extension of the spine. Forces on the facets increase with extension.

Lumbar facet joints also restrict twisting of the spine. Naturally, the force on the facet joints also increases with twisting or rotation of the spine. The prior-art devices do not dampen the rotational forces applied to the spine. Thus, low back pain from rotational forces on arthritic facet joints is not prevented with prior art devices.

### SUMMARY OF THE INVENTION

This invention improves upon the prior art through the addition of cross-coupled members to help prevent rotational forces on the facet joints, with particular emphasis on the posterior portion of the lumbar spine. Rigid, semi-rigid, or elastic members may be used depending upon the desired degree of resistance.

The cross-coupled members may assume different forms, including cables and polymer, fibrous, or elastic bands. For example, vertebral motion may be damped by connecting the screws with elastic bands. Vertebral motion could be further damped by covering the anterior bands with rubber or elastomeric sleeves similar to the sleeves used over the posterior bands of the prior art devices described above.

Although the configuration may be used as an adjunct to spinal fusion, it may also be used to dampen motion as an adjunct to vertebral anthroplasty.

### BRIEF DESCRIPTION OF THE DRAWINGS

FIG. 1A is an anterior view of a cable-based cross-coupled vertebral stabilizing mechanism according to U.S. Pat. No. 6,248,106;

FIG. 1B is a drawing which shows the mechanism of FIG. 1A from a lateral perspective;

FIG. 2 is a drawing which shows how cable-receiving discs may be stacked to join three or more vertebrae;

FIG. 3 is a drawing which shows how different types of cable-holding devices may be combined to join multiple vertebra;

FIG. 4 shows the use of preformed sleeves;

FIG. 5 depicts the use of additional devices for protecting cables from abrading one another where they cross;

FIG. 6 is a drawing which illustrates the alternative use of a centerpiece with four cables attached thereto using screws or alternative fasteners;

FIG. 7 is a drawing which illustrates the alternative use of tumbuckles on one or more cables;

FIG. 8 is a view in perspective of different elements constituting a stabilization device according to U.S. Pat. No. 5,540,688, to which the instant invention is applicable;

FIG. 9 is a view from behind of three vertebrae associated with the stabilization devices of FIG. 8;

FIG. 10 is a section along III--III of FIG. 9;

FIG. 11 is a posterior view of a prior-art vertebral stabilizing mechanism including cross-coupled stabilization according to the invention; and

FIG. 12 illustrates an attachment arrangement other than pedicle screws.

### DETAILED DESCRIPTION OF THE INVENTION

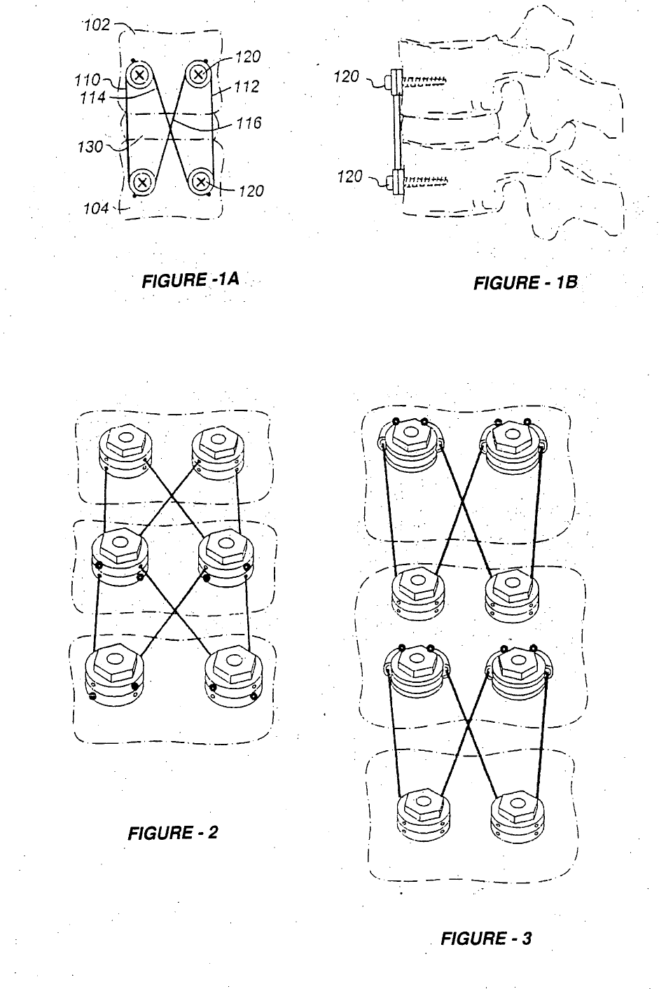

FIG. 1A is an anterior view of a cable-based cross-coupled vertebral stabilizing mechanism disclosed in U.S. Pat. No. 6,248,106, incorporated herein by reference. FIG. 1B is a drawing which shows the mechanism of FIG. 1A from a lateral perspective. In this illustration, the mechanism is used to join upper and lower vertebrae 102 and 104, respectively, though the mechanism is applicable to multiple levels, as shown in FIGS. 2 and 3. Note that some form of intervertebral cage and/or bone graft 130 may be used in between the vertebrae 102 and 104 to resist compression.

Broadly, the mechanism utilizes a pair of fasteners on each vertebrae, and elongated elements, preferably cables, in an axial and criss-crossed pattern to provide an arrangement that resists extension, lateral bending, and torsional/rotational stresses. As best seen in FIG. 1A, a preferred configuration utilizes a pair of screws 120 in the upper vertebrae, and a corresponding pair in the lower vertebrae, along with a pair of longitudinal cables 110 and 112, which are used in conjunction with a pair of criss-cross cables 114 and 116.

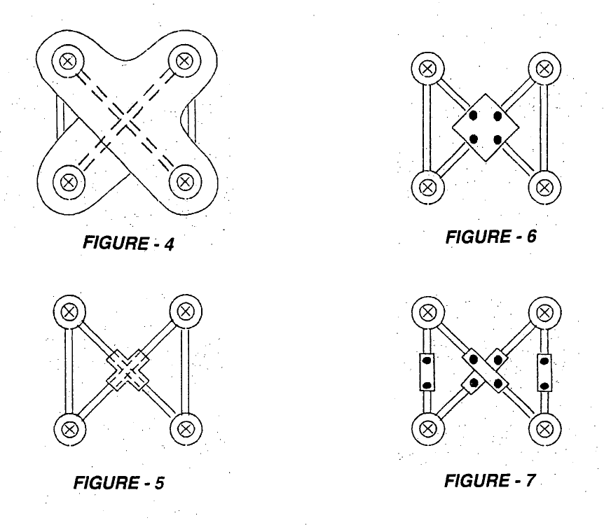

FIG. 2 is a drawing which shows how cable-receiving discs may be stacked to 20 join three or more vertebrae. FIG. 3 shows how different types of cable-holding devices may be combined to join multiple vertebra. Such devices may be covered with soft materials such as silastic in various ways. For example, preformed sleeves may be placed over prominent portions of the device, as shown in FIG. 4. Alternatively, liquid polymer may be poured over, or injected to surround the device. The material could be strengthened by inserting fibers into and around the device before or during the pouring or injection procedure. Polymer would be selected on the basis that it would cure rapidly and safely within the body.

Additional devices may be provided to protect the cables from abrading one another where they cross in the middle. For example, an x-shaped device with holes could be placed over the crossing wires, as shown in FIG. 5. Preferably, the wires would cross over the device in different planes to prevent friction with one another. Alternatively, a centerpiece could be used wherein four cables attached thereto using screws or alternative fasteners (FIG. 6). As yet a further alternative, as shown in FIG. 7, turnbuckles may be incorporated into the cables or threaded rods to tighten them during installation or, perhaps as part of a postoperative or revision procedure.

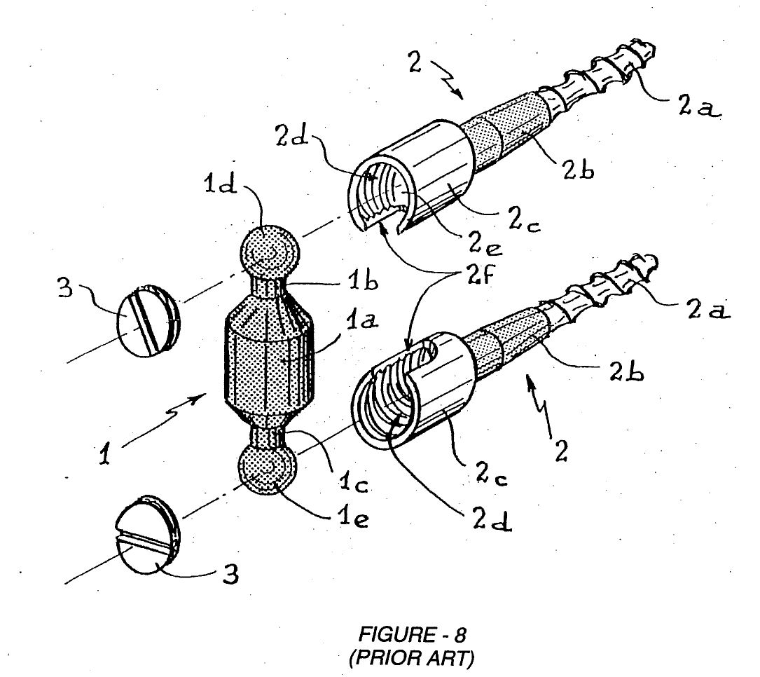

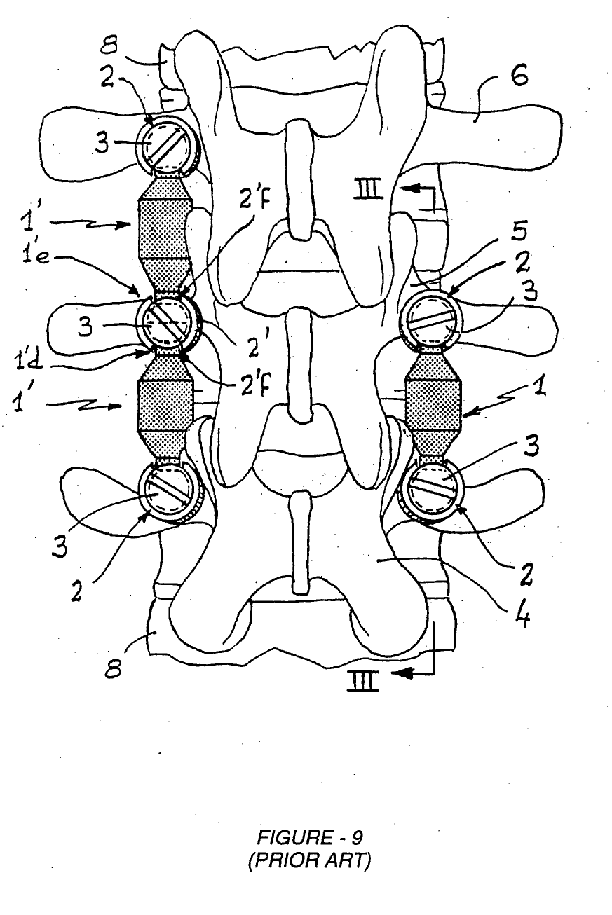

FIG. 8 is a view in perspective of different elements constituting a stabilization device according to U.S. Pat. No. 5,540,688, the entire content of which is incorporated herein by reference. The instant invention is applicable this device as well as to any other apparatus which provides two or more spinally aligned intervertebral stabilization devices, particularly those installed using pedicle screws and including dampers, as disclosed in U.S. Pat. Nos. 5,375,823; 5,480,401; 5,584,834; 5,591,166; 5,628,740; 5,961,516; EP 576379; EP 611554; EP 667127, and FR 2697428, all of which are incorporated herein by reference.

The device of U.S. Pat. No. 5,540,688 essentially comprises a damper I made of a bio-compatible, elastic material and two implants 2 screwed in two adjacent vertebrae and whose free ends are associated with the two ends of the damper 1. It is observed that the damper 1 is made in the form of an elongated body provided with a bulged or enlarged central part 1a joined to two necks 1b, 1c to two bulbous ends 1d, 1e. In an advantageous embodiment of the preceding arrangement, the bulged part 1a may be provided to be of elliptic longitudinal section, while the two ends 1d and 1c each take the form of a sphere. Of course, the part 1a may be of cylindrical section with two truncated endpieces or in the form of two frustums of cone or may be asymmetrical in particular applications.

Each implant 1 includes a screw 2a adapted to be screwed in the pedicle of a vertebra or in any other location thereof. The screw 2a extends from a cylindrical body 2b which terminates in a hollow socket or receptacle 2c of cylindrical shape with a tapped inner wall 2d and a concave bottom 2e presenting a shape complementary to that of half the end 1d, 1e of the damper. It is observed that the socket 2c is provided with a lateral notch 2f adapted to allow passage of the neck 1b, 1c of the damper 1 for positioning the damper with respect to the implants. Locking of the ends of the damper 1 is effected after they have been placed in the sockets 2c by screwing a threaded endpiece 3 inside the corresponding socket with respect to the tapped wall 2d. Of course, the base 3a of the endpiece 3 is provided to be concave and hemi-spherical, so as to cooperate exactly with the spherical ends 1d, 1e of the damper.

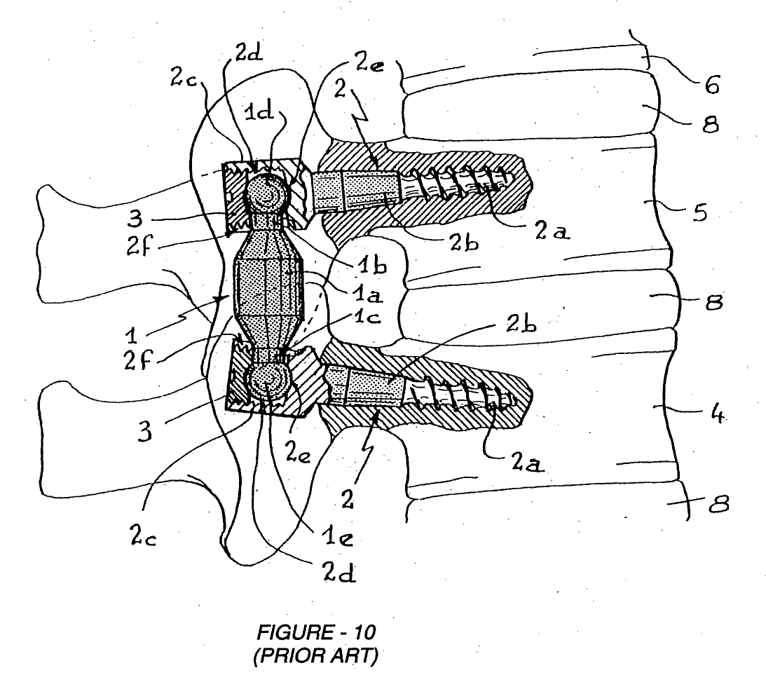

FIGS. 9 and 10 illustrate the assembly of a device according to the invention with respect to two adjacent vertebrae 4 and 5 of a spine. On the right-hand side of FIG. 9, a device has been illustrated, comprising one damper 1 associated with two implants 2 each fastened to a vertebra 4, 5. The same assembly may be provided in the left-hand part. In addition, it is possible that three successive vertebrae 4, 5, 6 need stabilization. In that case, one of the implants 2′ comprises two diametrically opposite notches 2f, while the ends of the two dampers 1′ each comprise one end 1′d, 1′e, truncated along a diametrical plane of the sphere perpendicular to the longitudinal axis of the damper in order that the two truncated ends 1′d, 1′e may be retained in the socket of the implant 2′ (cf. the left-hand part of FIG. 9).

FIG. 10 shows in very detailed manner the structure of the assembly of the ends of the damper with two implants. The hollow socket 2c with bellied concave base 2e is found again, as well as the endpiece 3 with bellied concave base 3a in order that the two spherical ends 1c, 1d of the damper 1 are suitably locked with respect to the implants 2. Such locking makes it possible to create a sort of ball joint articulation facilitating the movements of the spine.

Accordingly, prior-art devices of the type just described do not dampen the rotational forces applied to the spine. Anatomically, the lumbar facet joints restrict twisting of the spine, and the force on the facet joints increases with increasing twisting and/or rotation. Thus, low back pain from rotational forces on arthritic facet joints is not prevented with these devices.

This invention improves upon the prior art through the addition of cross-coupled members to help prevent rotational forces on the facet joints, with particular emphasis on the posterior portion of the lumbar spine. The cross-coupled members may assume different forms, including cables and polymer, fibrous, or elastic bands. Although the configuration may be used as an adjunct to spinal fusion, it may also be used to dampen motion as an adjunct to vertebral anthroplasty.

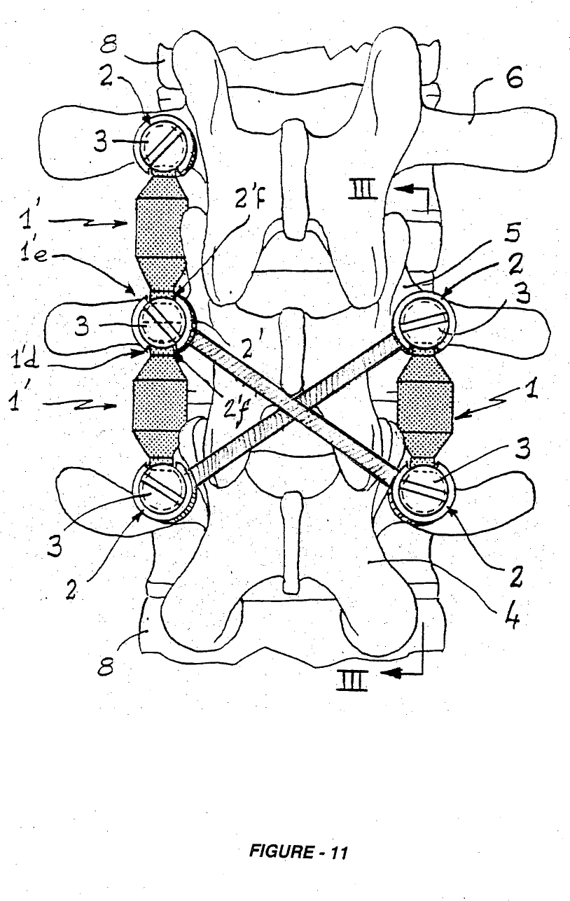

FIG. 11 is a posterior view of the prior-art vertebral stabilizing mechanism of FIGS. 8 through 10, but including cross-coupled stabilization according to this invention. Rigid, semi-rigid, or elastic members may be used depending upon the desired degree of resistance. For example, vertebral motion may be damped by connecting the screws with elastic bands. Vertebral motion could be further damped by covering the anterior bands with rubber sleeves similar to the sleeves used over the posterior bands of the prior art devices described above.

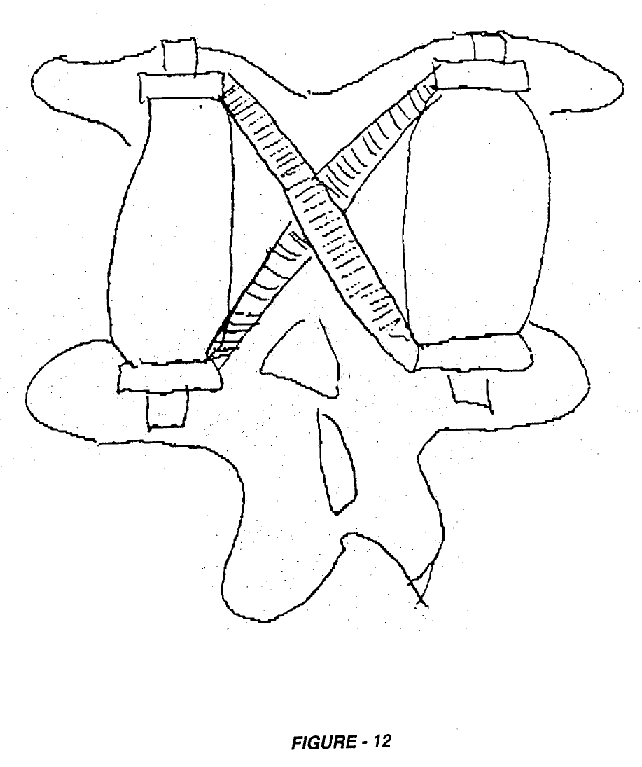

The cross-coupling elements according to the invention need not attach with pedicle screws. FIG. 12 illustrates an alternative configuration wherein the ends of the cross-coupling elements attached more directly to dampening elements. In addition, although in the preferred embodiment the cross-coupled elements attach at the points where the dampening elements attach, this is not essential to the invention, since the ends of the cross-coupling elements may attach at separate points while still providing resistance to twisting and/or rotational motion.

What is Claimed is:

1. A method for stabilizing upper and lower spinal vertebrae having a disc space situated therebetween, comprising the steps of:
inserting first and second fasteners into the upper vertebra;
inserting third and fourth fasteners into the lower vertebra, wherein the first and third fasteners are substantially vertically aligned and the second and fourth fasteners are substantially vertically aligned;
connecting the first and third fasteners with a first elongate element;
connecting the second and fourth fasteners with a second elongate element;
connecting the first and fourth fasteners with a third elongate element; and
connecting the second and third fasteners with a fourth elongate element.

2. The method of claim 1, wherein the first, second, third, and fourth elongate elements are cables.

3. The method of claim 1, wherein the first, second, third, and fourth elongate elements are elastic connectors.

4. The method of claim 1, wherein the first, second, third, and fourth fasteners are screws.

5. The method of claim 1, wherein at least a portion of the first, second, third, and fourth elongate elements are covered.

6. The method of claim 1, wherein at least one of the first, second, third, and fourth elongate elements are bands.

7. The method of claim 1, wherein at least one of the first, second, third, and fourth elongate elements are elastic.

8. The method of claim 1, wherein at least one of the first, second, third, and fourth elongate elements are fibrous.

9. The method of claim 1, wherein at least one of the first, second, third, and fourth elongate elements comprise polymers.

10. The method of claim 1, further comprising the step of inserting an object between the upper and lower spinal vertebrae that resists compression.

11. The method of claim 10, wherein the object is an intervertebral cage.

12. The method of claim 10, wherein the object is a distracting plug.

13. The method of claim 1, further comprising the step of inserting bone graft into the disc space between the upper and lower spinal vertebrae.

14. The method of claim 1, wherein the first, second, third, and fourth elongate elements pull at least two of the first, second, third, and fourth fasteners closer together.

15. The method of claim 1, wherein compression is applied across the disc space.

16. The method of claim 1, further comprising the step of tightening at least one of the first, second, third, and fourth elongate elements.

17. An apparatus for stabilizing upper and lower spinal vertebrae having a disc space situated therebetween, comprising:
a first pair of right and left fasteners adapted for attachment to the upper vertebra;
a second pair of right and left fasteners adapted for attachment to the lower vertebra;
a first elongate element interconnecting the right fasteners of the first and second pair of fasteners;
a second elongate element interconnecting the left fasteners of the first and second pair of fasteners;
a third elongate element interconnecting the right fastener of the first pair and the left fastener of the second pair; and
a fourth elongate element interconnecting the left fastener of the first pair and the right fastener of the second pair.

18. The device of claim 17, further comprising an intervertebral cage adapted for placement into the disc space.

19. The device of claim 17, further comprising bone graft adapted for placement into the disc space.

20. The device of claim 17, wherein at least one of the first, second, third, and fourth elongate elements are bands.

21. The device of claim 17, wherein at least one of the first, second, third, and fourth elongate elements are elastic.

22. The device of claim 17, wherein at least one of the first, second, third, and fourth elongate elements are fibrous.

23. The device of claim 17, wherein at least one of the first, second, third, and fourth elongate elements comprise polymers.
